(19) United States
(12) Patent Application Publication
(10) Pub. No.: US 2009/0305239 A1
(43) Pub. Date: Dec. 10, 2009

(54) Methods and compositions for determing a level of biologically active serum paraoxonase

(76) Inventors: Dan S. Tawfik, Zur-Hadassa (IL); Olga Khersonsky, Jerusalem (IL)

Correspondence Address: MARTIN D. MOYNIHAN d/b/a PRTSI, INC., P.O. BOX 16446, ARLINGTON, VA, 22215, US

(21) Appl. No.: 11/990,393

(22) PCT Filed: Aug. 14, 2006
(86) PCT No.: PCT/IL2006/000941

§ 371 Date: Feb. 5, 2009

Related U.S. Application Data
(60) Provisional application No. 60/708,767, filed on Aug. 17, 2005.

(51) Int. Cl. C12Q 1/68; C12Q 1/44; C12Q 1/00; C07D 313/04; C07D 309/30; C07D 307/02; G01N 27/26

(52) U.S. Cl. 435/6; 435/19; 435/4; 549/263; 549/266; 549/273; 549/295; 205/777.5

(57) ABSTRACT

A method of determining a level of biologically active PON enzyme is provided. The method comprising determining lactonase activity of the PON enzyme, the lactonase activity being indicative of the level of biologically active PON enzyme. Also provided are novel compounds which may be used for measuring a lactonase activity of an enzyme.

### FIELD AND BACKGROUND OF THE INVENTION

The present invention relates to a biochemical diagnosis and, more particularly, to methods and compositions for determining a level of biologically active serum paraoxonase (PON), such as PON1.

Serum paraoxonase (PON1) is the most familiar member of a large family of enzymes dubbed PONs. PON1 is an HDL-associated enzyme with anti-atherogenic and detoxification properties that hydrolyzes a wide range of substrates, such as esters, organophosphates (e.g., paraoxon) and lactones. For a long time, PON1 was considered an aryl-esterase and paraoxonase, and its activity was measured accordingly. However, it recently became apparent that PON1 is primarily a lactonase catalyzing both the hydrolysis^{[1, 2]} and formation^{[3]} of a variety of lactones. Structure-reactivity studies^{[4]} and laboratory evolution experiments^{[5]} indicate that PON1's native activity is lactonase, and that the paraoxonase and aryl esterase are promiscuous activities. Studies of PON1's activation by binding to HDL particles carrying ApoA-I indicate high specificity towards lactone substrates, and lipophilic lactones in particular^{[6]}. Finally, the lactonase activity is the only activity shared by all members of the PON family, some of which exhibit no paraoxonase or aryl esterase activity^{[2]}.

The activity of PON1 in human sera has been the subject of numerous studies that address a possible linkage between the polymorphism of PON1, various environmental factors that modulate its activity, and susceptibility to atherosclerosis and other disorders^{[7]}. The assays, however, use phenyl acetate or paraoxon that have no physiological relevance. A more relevant assay must address the lactonase activity. Current methods for measuring lactonase activities with aliphatic lactones are based on pH indicators^{[1, 4]} and HPLC^{[2, 3]}. The latter is highly laborious, while the pH indicator assay requires repetitive calibrations and gives accurate results only with pure enzymes samples where the pH and buffer strength can be tightly controlled.

Recently, Sicard and co-workers^{[9]} developed a fluorescence-based lactonase assay using 6- and 7-membered ring lactones substituted with umbelliferone. However, these substrates significantly differ from the favorable substrates of PON1 that comprise 5-membered ring oxo-lactones with long alkyl side-chains^{[2, 4, 6]}. These substrates also exhibit high background rates at the pH optimum for PON1 (8.0-8.5).

There is thus a widely recognized need for, and it would be highly advantageous to have, a novel assay for lactonase activity which is devoid of the above limitations.

### SUMMARY OF THE INVENTION

According to one aspect of the present invention there is provided a method of determining a level of biologically active PON enzyme, the method comprising determining lactonase activity of the PON enzyme, the lactonase activity being indicative of the level of biologically active PON enzyme.

According to another aspect of the present invention there is provided a method of determining PON status in a subject, the method comprising: (a) determining lactonase activity level of a PON enzyme of the subject, the lactonase activity being indicative of the level of biologically active PON in the subject; and (b) genotyping the PON enzymes of the subject, thereby determining PON status of the subject.

According to still further features in the described preferred embodiments the PON enzyme is selected from the group consisting of PON1, PON2 and PON3.

According to still further features in the described preferred embodiments the biologically active PON enzyme comprises apolipoprotein complexed PON enzyme.

According to still further features in the described preferred embodiments determining lactonase activity of the PON enzyme is effected by:

(i) a chromatographic analysis;
(ii) a pH indicator assay;
(iii) a spectrophotometric assay;
(iv) a coupled assay;
(v) an electrochemical assay; and/or
(vi) a therm-ocalometric assay.

According to still further features in the described preferred embodiments the spectrophotometric assay is effected in the presence of a substrate comprising at least one lactone and being capable of forming at least one spectrophotometrically detectable moiety upon hydrolysis of the lactone.

According to still further features in the described preferred embodiments the spectrophotometric assay is selected from the group consisting of a phosphorescence assay, a fluorescence assay, a chromogenic assay, a luminescence assay and an illuminiscence assay.

According to still further features in the described preferred embodiments the detectable moiety is attached to the lactone.

According to still further features in the described preferred embodiments the detectable moiety forms a part of the lactone.

According to still further features in the described preferred embodiments the detectable moiety comprises at least one thiol.

According to still further features in the described preferred embodiments the substrate comprises a thioalkoxy group being attached to the lactone.

According to still further features in the described preferred embodiments the thioalkoxy group comprises from 2 to 12 carbon atoms.

According to still further features in the described preferred embodiments the detecting is effected by a chromogenic assay or a fluorogenic assay.

According to still further features in the described preferred embodiments the substrate comprises a 5-, 6- or 7-membered lactone having a thioalkoxy group attached to the carbon adjacent to the heteroatom of the lactone.

According to yet another aspect of the present invention there is provided a method of determining activity of a lactonase in a sample comprising: (a) contacting the sample with a compound containing at least one lactone and being capable of forming at least one spectrophotometrically detectable moiety upon hydrolysis of the lactone, wherein the detectable moiety is selected such that the compound has substantially the same structure as a substrate of the lactonase; and (b) spectrophotometrically measuring a level of the moiety, thereby determining an activity of the lactonase in the sample.

According to still further features in the described preferred embodiments measuring the level of the moiety is effected by a phosphorescence assay, a fluorescence assay, a chromogenic assay, a luminescence assay and an illuminiscence assay.

According to still further features in the described preferred embodiments the detectable moiety is attached to the lactone.

According to still further features in the described preferred embodiments the detectable moiety forms a part of the lactone.

According to still further features in the described preferred embodiments the detectable moiety comprises at least one thiol.

According to still further features in the described preferred embodiments the substrate comprises a thioalkoxy group being attached to the lactone.

According to still further features in the described preferred embodiments the thioalkoxy group comprises from 2 to 12 carbon atoms.

According to still further features in the described preferred embodiments the detecting is effected by a chromogenic assay.

According to still another aspect of the present invention there is provided a kit for determining predisposition or diagnosing a disorder associated with abnormal levels or activity of a PON enzyme in a subject, the kit comprising at least one agent capable of determining lactonase activity of the PON enzyme.

According to still further features in the described preferred embodiments the at least one agent is a compound comprising at least one lactone and being capable of forming at least one spectrophotometrically detectable moiety upon hydrolysis of the lactone.

According to an additional aspect of the present invention there is provided a compound comprising at least one lactone and being capable of forming at least one spectrophotometrically detectable thiol-containing moiety upon decomposition of the lactone.

According to still further features in the described preferred embodiments thiol-containing moiety is detectable by a spectrophotometric assay selected from the group consisting of a phosphorescence assay, a fluorescence assay, a chromogenic assay, a luminescence assay and an illuminiscence assay.

According to still further features in the described preferred embodiments the detectable moiety is attached to the lactone.

According to still further features in the described preferred embodiments the detectable moiety forms a part of the lactone.

According to still further features in the described preferred embodiments the detectable moiety comprises a thioalkoxy group.

According to still further features in the described preferred embodiments the thioalkoxy group comprises from 2 to 12 carbon atoms.

According to still further features in the described preferred embodiments the lactone is a 5-, 6- or 7-membered lactone.

According to still further features in the described preferred embodiments the lactone is a five-membered lactone.

The present invention successfully addresses the shortcomings of the presently known configurations by providing methods and compositions for determining a level of biologically active serum paraoxonase.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are graphs showing calorimetric (FIG. 1a) and fluorogenic (FIG. 1b) measurements of the lactonase activity of PON1. FIG. 1a—0.2 mM TBBL with 0.5 mM DTNB, in the presence of PON1 (8.375×10⁻⁹ M; closed squares) or its absence (opened circled), monitored by absorbance at 412 nm. FIG. 1b—0.25 mM TBBL with 50 μM CPM, in the presence of PON1 (8.375×10⁻⁹ M; closed squares) or its absence (opened circles), detected by excitation at 400 nm and emission at 516 nm.

FIGS. 2a-b are graphs showing lactonase (FIG. 2a) and aryl esterase (FIG. 2b) activities of PON1 in human sera. Sera were diluted 1:400 in Tris pH 8.0, and reactions included: FIG. 2a—0.5 mM TBBL and 0.5 mM DTNB; FIG. 2b—1.0 mM phenyl acetate. Shown are the rates observed with no inhibitor (closed circles), with 100 μM 2-hydroxyquinoline (opened circles), or 5 mM EDTA (closed triangles), and the background hydrolysis with no serum (opened squares). Hydrolysis of TBBL was detected with DTNB and monitored by absorbance at 412 nm (FIG. 2a). Hydrolysis of phenyl acetate was monitored directly by absorbance at 270 nm (FIG. 2b).

FIG. 3 is a graph showing PON1 lactonase activity in PON1—expressing E. coli using a thio-alkyl butyrolactone substrate (TBBL) and w/o/w emulsions, as determined by FACS analysis. Cells expressing rePON1 in their cytoplasm were emulsified, together with TBBL and the thiol-detecting dye CPM. Shown are representative histograms of the fluorescent emission at 530 nm (the thiol-CPM adduct) for single cells expressing GFP and PON1 (white), and control cells with GFP only (grey).

### DESCRIPTION OF THE PREFERRED EMBODIMENTS

The present invention is of methods and compositions for determining a level of biologically active lactonases, and more specifically serum paraoxonase, a novel family of synthetic substrates thereof and methods of preparing same.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Paraoxonase 1 (PON1) is a member of a family of proteins that also include PON2 and PON3. PON1 is an HDL-associated enzyme with anti-atherogenic and detoxification properties that hydrolyzes a wide range of substrates, such as esters, organophosphates (e.g., paraoxon) and lactones. For a long time, PON1 was considered an aryl-esterase and paraoxonase, and its activity was measured accordingly. However, it recently became apparent that PON 1 is primarily a lactonase catalyzing both the hydrolysis and formation of a variety of lactones. Structure-reactivity studies and laboratory evolution experiments indicate that PON1's native activity is lactonase, and that the paraoxonase and aryl esterase are promiscuous activities.

The current convention suggests that it is the catalytic efficiency with which PON1 degrades toxic organophosphates and metabolizes oxidized lipids that determines the degree of protection provided by PON1 against physiological or xenobiotic toxins, i.e., chemical compounds which are foreign to the body or to living organisms. In addition, higher concentrations of PON1 provide better protection.

Thus, for adequate risk assessment it is important to know PON levels and activity.

While as mentioned hereinabove, lactonase activity of PON has been recently uncovered, analysis of PONs lactonase activity for faithfully assessing PONs biological activity has never been suggested.

While reducing the present invention to practice, the present inventors uncovered that determining lactonase activity of PON can be used for determining the level of biologically active PON in individuals. These findings may facilitate accurate risk assessment to numerous conditions associated with PON under-activity or levels, such as atherosclerosis.

Thus, according to one aspect of the present invention, there is provided a method of determining a level of biologically active PON enzyme.

As used herein the phrase “PON enzyme” refers to a paraoxonase enzyme (e.g., mammalian paraoxonase) such as human PON1 (GenBank Accession No. NP_{—}000437.3), human PON2 (GenBank Accession No. NP_{—}000296.1) and human PON3 (GenBank Accession No. NP_{—}000931.1).

As used herein the phrase “biologically active PON enzyme” refers to the fraction of PON enzyme which is involved in biological (e.g., physiological) events, such as for example, hydrolysis of oxidized lipids.

For example, biologically active PON enzyme can refer to the fraction of PON enzyme which is associated with various apolipoprotein particles, such as HDL-apoA-I. It has recently been established that PON enzyme associated with apoA-I is capable of stimulating higher PON lactonase activity as compared to apoA-IV and apoA-II [see Gaidukov and Tawfik (2005) Biochemistry In-press).

Preferably, PON enzymes of the present invention are present in biological samples derived from an animal subject (e.g., human), such as further described hereinbelow.

The method of this aspect of the present invention is effected by determining lactonase activity of the PON enzyme, such lactonase activity being indicative of the level of biologically active PON enzyme.

As used herein the phrase “lactonase activity” refers to lactone hydrolysis activity, which typically, in accordance with this aspect of the present invention, refers to the hydrolysis of an ester bond of a lactone.

Methods of determining a lactonase activity of an enzyme are well known in the art. These methods are typically effected by known biochemical assays such, for example, chromatrographic assays (e.g., HPLC, TLC, GC, CPE) pH indicator assays, coupled assays (i.e., in these assays enzymes other than the one assayed are added to yield a measurable product; For example, the carboxylic acid product could be turned over by a dehydrogenase, and the change in concentration of NAD/NADH, or NADP/NADPH, monitored by absorbance or fluoresecence), therm-ocalorimetric (i.e., monitoring changes in heat capacity), electrochemical assays (i.e., monitoring changes in redox potential) and/or spectrophotometric assays.

A typical enzyme assay is based on a chemical reaction which the tested enzyme catalyzes specifically. The chemical reaction is typically the conversion of a substrate or an analogue thereof into a product. The ability to detect minute changes in the levels, i.e., the concentration of either the substrate or the product enables the determination of the enzyme's activity both qualitatively and quantitatively, and even quantitatively determines the specificity of a particular substrate to the tested enzyme. In order to measure minute changes in the levels of the substrate and/or the product, these compounds should have a chemical and/or physical property which can be detected chemically or physically, such as a change in pH, molecular weight, color or another directly or indirectly measurable chemical and/or physical property.

Following is a description of exemplary lactonase assays which can be used in accordance with this aspect of the present invention.

pH indicator assays—Enzymatic assays which are based on pH indicators are typically used for measuring lactonase activity with aliphatic lactones. This may be achieved using the continuous pH-sensitive colorimetric assay (i.e., measuring the intensity of color generated by a pH indicator) such as described in Billecke et al. (2000) Drug Metab. Dispos. 28:1335-1342, using a SPECTRAmax® PLUS microplate reader (Molecular Devices, Sunnyvale, Calif.). The reactions (200 μl final volume) containing 2 mM HEPES, pH 8.0, 1 mM CaCl₂, 0.004% (w/v) Phenol Red, and diluted/non-diluted PON containing sample (e.g., serum sample, diluted 100-1000 fold) are initiated with 2 μl of 100 mM substrate solution in methanol and are carried out at 37° C. for 3-10 minutes. The rates are calculated from the slopes of the absorbance decrease at 558 nm with correction at 475 nm (iososbestic point) using a rate factor (mOD/μmol H⁺) estimated from a standard curve generated with known amounts of HCL. The spontaneous hydrolysis of the lactones and acidification by atmospheric CO₂ are preferably corrected for by carrying out parallel reactions with the same volume of storage buffer instead of enzyme.

Alternatively, proton release resulting from carboxylic acid formation can be monitored using the pH indicator cresol purple. The reactions are performed at pH 8.0-8.3 in bicine buffer 2.5 mM, containing 1 mM CaCl₂ and 0.2 M NaCl. The reaction mixture contains 0.2-0.3 mM cresol red (from a 60 mM stock in DMSO). Upon mixture of the substrate with the enzyme sample, the decrease in absorbance at 577 nm is monitored in a microtiter plate reader. The assay requires in situ calibration with acetic acid (standard acid titration curve), which gives the rate factor (−OD/mole of H⁺).

HPLC analysis—Hydrolysis of various lactone substrates can be detected by HPLC analysis. Thus for example, the hydrolysis of acylhomoserine lactones (AHLs) can be analyzed by HPLC (e.g., Waters 2695 system equipped with Waters 2996 photodiode array detector set at 197 nm using Supelco Discovery C-18 column (250×4.6 mm, 5 μm particles). Enzymatic reactions are carried at room temperature in 50 μl volume of 25 mM Tris-HCl, pH 7.4, 1 mM CaCl₂, 25 μM AHL (e.g., from 2 mM stock solution in methanol) and diluted/non-diluted PON containing sample (e.g., serum sample, diluted 100-1000 fold). Reactions are stopped with 50 μl acetonitrile (ACN) and centrifuged to remove the protein. Supernatants (40 μl) are loaded onto an HPLC system and eluted isocratically with 85% CAN/0.2% acetic acid (tetradeca-homoserine lactone). 0.75% CAN/0.2% acetic acid (dodeca-homoserine lactone), 50% CAN/)0.2% acetic acid (hepta-homoserine lactone), or 20% CAN/0.2% acetic acid (3-oxo-hexanoyl homoserine lactone).

The hydrolysis of the statin lactones (mevastatin, lovastatin and simvastatin) can be analyzed by high performance liquid chromatography (HPLC) such as by using a Beckman System Gold HPLC with a Model 126 Programmable Solvent Module, a Model 168 Diode Array Detector set at 238 nm, a Model 7125 Rheodyne manual injector valve with a 20 μl loop, and a Beckman ODS Ultrasphere column (C 18, 250×4.6 mm, 5 μm). Lovastatin (Mevacor) and simvastatin can be purchased as 20 mg tablets from Merck, from which the lactones are extracted with chloroform, evaporated to dryness and redissolved in methanol. Mevastatin can be purchased from Sigma.

In a final volume of 1 ml, 10-200 μl of enzyme solution and 10 μl of substrate solution in methanol (0.5 mg/ml) are incubated at 25° C. in 50 mM Tris/HCl (pH 7.6), 1 mM CaCl₂. Aliquots (100 μl) are removed at specified times and added to acetonitrile (100 μl), vortexed, and centrifuged for one minute at maximum speed (Beckman microfuge). The supernatants are poured into new tubes, capped and stored on ice until HPLC analysis.

Samples are eluted isocratically at a flow rate of 1.0 ml/min with a mobile phase consisting of the following: A=acetic acid/acetonitrile/water (2:249:249, v/v/v) and B=acetonitrile, in A/B ratios of 50/50, 45/55 and 40/60 for mevastatin, lovastatin and simvastatin, respectively.

Spectrophotometric assays—In these assays the consumption of the substrate and/or the formation of the product can be measured by following changes in the concentrations of a spectrophotometrically detectable moiety that is formed during the enzymatic catalysis. Examples of spectrophotometric assays include, without limitation, phosphorescence assays, fluorescence assays, chromogenic assays, luminescence assays and illuminiscence assays.

Phosphorescence assays monitor changes in the luminescence produced by a spectrophotometrically detectable moiety after absorbing radiant energy or other types of energy. Phosphorescence is distinguished from fluorescence in that it continues even after the radiation causing it has ceased.

Fluorescence assays monitor changes in the luminescence produced by a spectrophotometrically detectable moiety under stimulation or excitation by light or other forms of electromagnetic radiation or by other means. The light is given off only while the stimulation continues; in this the phenomenon differs from phosphorescence, in which light continues to be emitted after the excitation by other radiation has ceased.

Chromogenic assays monitor changes in color of the assay medium produced by a spectrophotometrically detectable moiety which has a characteristic wavelength.

Luminescence assays monitor changes in the luminescence produced a chemiluminescent and therefore spectrophotometrically detectable moiety generated or consumed during the enzymatic reaction. Luminescence is caused by the movement of electrons within a substance from more energetic states to less energetic states.

The phrase “spectrophotometrically detectable” as used in the context of the present invention describes a physical phenomena pertaining to the behavior of measurable electromagnetic radiation that has a wavelength in the range from ultraviolet to infrared. Non-limiting examples of spectrophotometrically detectable properties which can be measured quantitatively are color, illuminance and infrared and/or UV specific signature of a chemical compound.

The phrase “spectrophotometrically detectable moiety” therefore describes a moiety, which is formed during an enzymatic assay, and which is characterized by one or more spectrophotometrically detectable properties, as defined hereinabove. The concentration of such a moiety, which correlates to the enzymatic activity, can thus be quantitatively determined during an enzymatic reaction assay.

As mentioned above, lactones are natural substrates of PON enzymes. Thus, in each of the above describes assays, the substrate preferably comprises one or more lactone moieties.

As is well known in the art, the term “lactone” describes a cyclic carboxylic moiety such as a cyclic ester, which is typically the condensation product of an intramolecular reaction between an alcohol and a carboxylic ester. The latter is oftentimes referred to in the art as “oxo-lactone”. The term “lactone” also typically refers to cyclic thiocarboxylic moieties, and thus include also condensation products of an intramolecular reactions between a thiol group and a carboxylic acid, an alcohol and a thiocarboxylic acid and a thiol group and a thiocarboxylic acid. Such lactones are oftentimes collectively referred to in the art as “thiolactones”.

As is further well known in the art, the size of the lactone ring typically ranges from 4 to 8 atoms. Due to ring tension and other thermodynamic considerations, the ring size of common lactones typically ranges from 5 to 7 atoms. Such lactones are also known as favorable substrates of PON enzymes.

Commonly used prefixes may be used to indicate the lactone ring size: beta-lactone describes a 4-membered ring lactone, gamma-lactone describes a 5-membered ring lactone and delta-lactone describes a 6-membered ring.

The term “lactone” as used herein thus encompasses oxo-lactones and thiolactones, as described hereinabove, having 4-8 atoms, and preferably 5-7 atoms, in the lactone ring. The lactone moiety can be substituted or unsubstituted. When substituted, one or more carbon atoms in the lactone ring can be substituted by one or more substituents such as, but not limited to, alkyl, alkenyl, cycloalkyl, aryl, heteroaryl (bonded through a ring carbon) or heteroalicyclic (bonded through a ring carbon), alkoxy, thioalkoxy, as these terms as defined hereinbelow, and the likes.

As used herein, the term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted.

The term “alkenyl” refers to an alkyl group which consists of at least two carbon atoms and at least one carbon-carbon double bond.

The term “cycloalkyl” describes an all-carbon monocyclic or fused ring (i.e., rings which share an adjacent pair of carbon atoms) group where one or more of the rings does not have a completely conjugated pi-electron system.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system.

The term “aryl” describes an all-carbon monocyclic or fused-ring polycyclic (i.e., rings which share adjacent pairs of carbon atoms) groups having a completely conjugated pi-electron system.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “thiol” and “thiohydroxy” refers to a —SH group.

The term “hydroxy” refers to a —OH group.

The term “alkoxy”, as used herein, refers to an —O-alkyl group, as defined herein.

The term “thioalkoxy”, as used herein, refers to an —S-alkyl group, as defined herein.

The lactone moiety described hereinabove, when used as a substrate in the above described enzymatic assays, can further form a part of substance. Thus, for example, the lactone moiety can form a part of a fatty acid, a steroid, and the like.

According to a preferred embodiment of the present invention, determining a lactonase activity of a PON enzyme is effected by a spectorphotometric assay. Such an assay, according to further preferred embodiments of the present invention, utilizes substrates that comprise one or more lactones and which are capable of forming one or more spectorophotometrically detectable moieties. The enzyme is contacted with such substrates and the amount of the detectable moiety is measured.

In one embodiment of the spectrophotmetric assay described herein, a substrate in which

the spectrophotometrically detectable moiety forms an integral part of the lactone is utilized. In such assays, the enzyme hydrolyzes the lactone and a spectrophotometrically detectable species is generated in the assay medium. The substrate, hence, is a pre-spectrophotometrically detectable substance having a pre-spectrophotometrically detectable moiety in its structure.

As used herein, the phrase “pre-spectrophotometrically detectable moiety or substance” is used to describes a moiety or a substance that is capable of forming a detectable moiety under certain conditions, herein, when subjected to an enzymatic reaction.

A spectrophotometrically detectable moiety that forms a part of the lactone-containing substrate is highly advantageous since such substrates maintain the natural chemical and spatial specificity of the substrate to its natural enzyme, and thereby maintain the natural chemical interactions between the enzyme and the substrate. Maintaining these interactions enable to study and determine the natural biological activity of the enzyme, and also allows for a biologically meaningful comparison between other chemical effectors of the enzyme such as natural and synthetic inhibitors.

In one embodiment of the spectrophotmetric assay described herein, a substrate in which the spectrophotometrically detectable moiety is attached to the lactone is utilized. Such substrates are selected such that a spectrophotometrically detectable moiety is typically released upon the enzymatic reaction performed in the assay.

According to a preferred embodiment of this aspect of the present invention, the spectrophotometrically detectable moiety comprises a thiol group.

Thiols are known as highly convenient detectable groups. A thiol assay, can be effected, for example, by using a spectrophotometric method based on the reduction of the pro-dye 5,5′-dithiobis(2-nitrobenzoic acid; DTNB, also known as Ellman's reagent [Ellman, G. L., 1959, Arch. Biochem. Biophys. 82, 70-77]) by thiol groups. This reaction generates a colored species which can be detected at 412 nanometer wavelength, as described hereinbelow and is further exemplified in the Examples section that follows.

As discussed hereinabove, a thiol group can form a part of the lactone in the substrates utilized in this embodiments. Thus, one or more of the lactone moieties in the substrate may have a sulfur atom in the lactone ring which upon enzymatic hydrolysis generates a thiol. As illustrated in Scheme I below, the thiol can be detected by its typical reaction with DTNB, as is detailed hereinabove.

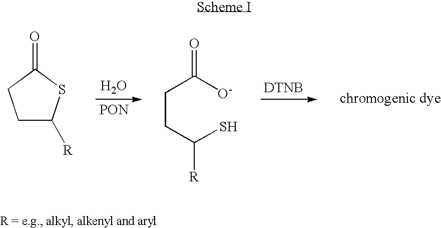

Optionally, a thiol-containing group can be attached to the lactone moiety in the substrate. Such thiol-containing substrates are designed such that a thiol-containing detectable moiety is released upon the enzymatic reaction. A preferred detectable moiety that comprises a thiol grouping this respect is a thioalkoxy group. The thioalkoxy group can be attached to the lactone such that upon enzymatic reaction, a thioalkyl is generated, as is illustrated in Scheme II below.

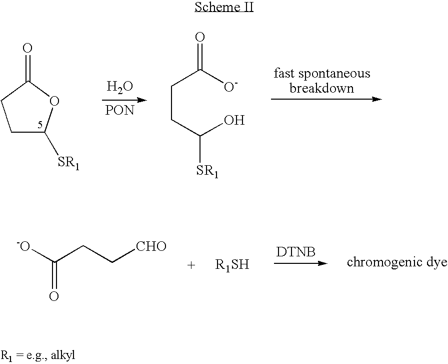

While further reducing the present invention to practice, the present inventors have designed and successfully prepared and used a series of novel lactone-containing compounds which may serve as efficient PON substrates in a lactonase activity assay.

Such lactone-containing compounds include one or more lactone rings, which upon decomposition thereof is capable of forming one or more spectrophotometrically detectable thiol-containing moiety and are collectively represented by the general Formula I:

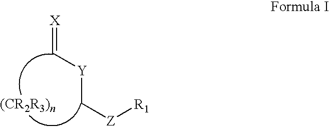

wherein X and Y are each an oxygen or a sulfur atom, Z is a carbon or a sulfur atom and at least one of Y and Z is a sulfur, n is an integer ranging between 2 and 4 and each of R₁, R₂ and R₃ are independently a hydrogen, an alkyl, alkenyl, cycloalkyl, aryl, heteroaryl (bonded through a ring carbon) or heteroalicyclic (bonded through a ring carbon), alkoxy and the likes.

The novel lactones can therefore be five-membered lactones, wherein n equals 2, sic-membered lactones, where n equals 3 or 7-membered lactones, where n equals 4. Preferably, n equals 2, forming a 5-membered lactone.

In one preferred embodiment, X and Y are both oxygen atoms and Z is a sulfur atom. Preferably, R₁ is an alkyl group having 2 to 12 carbon atoms.

Such a lactone typically undergoes lactonase-driven enzymatic hydrolysis by PON and thereafter releases a thiol as a result of a fast and spontaneous decomposition of the geminal thioalkoxy/thiohydroxy-hydroxy moiety which is formed in the hydrolysis. As illustrated in Scheme II above, the resulting thiol may be detected by a typical reaction with the DTNB as described hereinabove and exemplified in the Example section that follows.

In another preferred embodiment, X is oxygen and Y is sulfur, such that the compound is a thiolactone. In this embodiment, Z can be either carbon or sulfur, preferably carbon, and R₁ can be a hydrogen, an alkyl, alkenyl, cycloalkyl, aryl, heteroaryl (bonded through a ring carbon) or heteroalicyclic (bonded through a ring carbon), alkoxy and the likes and is preferably an alkyl having 2-12 carbon atoms. Such thiolactones can undergo a lactonase-driven enzymatic hydrolysis by PON, which generates a thiol group that can be subsequently detected.

The use of five-membered lactones that have an alkyl group or a thioalkoxy group attached at position 5 thereof in PON assays is highly advantageous since these compounds are almost identical to the favorable substrates of PON1, which comprise 5-membered ring oxo-lactones with long alkyl side-chains^{[2, 4, 6]}.

The thiol-containing moiety (e.g., a thioalkyl) generated in the enzymatic reaction may serve as a spectrophotometrically detectable moiety in, for example, phosphorescence assays, fluorescence assays, chromogenic assays, luminescence assays and illuminiscence assays, as discussed hereinabove, which are typically relatively simple and rapid techniques for detection and quantification of enzymatic activity.

As demonstrated and exemplified hereinbelow, the present inventors have used a series of lactone substrates having a spectrophotometrically detectable thioalkoxy moiety attached to a 5-membered ring lactone at position 5 thereof. As presented in the Examples section hereinbelow, the following lactones: 5-ethylsulfanyl-dihydro-furan-2-one, 5-butylsulfanyl-dihydro-furan-2-one and 5-hexylsulfanyl-dihydro-furan-2-one were prepared. These lactones, presented in Table 1 hereinbelow, exhibited k_{cat}/K_{M} values ranging between 1.5×10⁵ to 4.45×10⁵ which are comparable to k_{cat}/K_{M} values observed with lactones, and are considered acceptable values for enzyme substrates.

The k_{cat}/K_{M} value of an enzymatic activity gives a measurement of the substrate specificity. It allows comparing the specificity of different substrates for a same enzyme or the comparison of catalysis rates with different enzymes converting the same substrate. This ratio has a unit of a second order rate constant and is then expressed as 1/(concentration×time). Although values ≧10⁸M⁻¹ sec⁻¹ have been observed with certain enzymes, substrates having a k_{cat}/K_{M} ratio in the range 10⁴-10⁶ M⁻¹ sec⁻¹ are considered to be good substrates, i.e., exhibit reasonable affinity, specificity and rapid turn-over in the enzymatic assay.

Lactones which form a detectable moiety upon an enzymatic reaction and which are structurally similar to physiological lactonase substrates, such as the novel lactones described hereinabove, can be utilized for determining an activity of a lactonase in a sample.

Hence, according to another aspect of the present invention, there is provided a method of determining activity of a lactonase in a sample. The method, according to this aspect of the present invention is effected by:

(a) contacting the sample with a compound containing one or more lactones, as defined hereinabove, and being capable of forming one or more spectrophotometrically detectable moiety, as defined hereinabove, upon hydrolysis of one or more of the lactones, wherein the detectable moiety is selected such that the compound has substantially the same structure as a substrate of the lactonase; and

(b) spectrophotometrically measuring a level of the spectrophotometrically detectable moiety, thereby determining an activity of the lactonase in the sample.

As used herein, the phrase “having substantially the same structure as a substrate of the lactonase” refers to a chemical structure of a synthetic substrate which is almost identical to the structure of the natural substrate, differs therefrom by relatively minor chemical and/or structural features such as the replacement of one or two atoms, elongation of a side chain and the likes.

As in the specific case of the lactonase activity assay presented hereinabove, the assay of any lactonase activity preferably uses spectrophotometric assay techniques such as phosphorescence assays, fluorescence assays, chromogenic assays, luminescence assays and illuminiscence assays, as discussed hereinabove, since these assays usually require widely available machines and measuring devices for determining minute changes in the concentrations of spectrophotometrically detectable moieties and other chemical entities.

Measuring the level of any lactonase activity is effected by following the concentration levels of a detectable moiety which is attached to the lactone, either by forming a part of the lactone ring or by being attached thereto as a substituent, as described in the example of the PON lactonase activity assays discussed hereinabove.

As in the example of the PON lactonase activity assays discussed herein, the detectable moiety preferably includes one or more thiol groups.

It should be noted that the above-described agents for determining lactonase activity may be included in kits for determining predisposition of diagnosing disorders or conditions associated with abnormal levels or activity of a lactonase such as, for example, a PON enzyme in a subject.

As used herein the term “subject” or “individual” refers to a subject (e.g., mammal), preferably a human subject which is suspected of suffering or is at a risk of having a disorder which is associated with abnormal levels or activity of a PON enzyme.

As used herein the term “diagnosing” refers to classifying a disease, a condition or a symptom, or to determining a severity of the disease, condition or symptom monitoring disease progression, forecasting an outcome of a disease and/or prospects of recovery.

As used herein the phrase “disorders or conditions associated with abnormal (high or low levels as compared to a control sample obtained from a healthy subject) levels or activity of a PON enzyme” refers to various pathological and physiological conditions and diseases in which PON (e.g., PON1) activity is altered (see e.g., Costa et al. (2005) Biochemical Pharmacology 69:541-550, and references therein). For example, it has been shown that serum PON1 activity is low in both insulin-dependent (type I) and non-insulin-dependent (type II) diabetes, Alzheimer's disease (Dantoine et al. 2002 Paraoxonase 1 activity: a new vascular marker of dementia? Ann N Y Acad. Sci. 2002 November; 977:96-101), as well as in various cardiac disorders, including arteriosclerosis [Costa et al. (2005); Mackness et al. (2004) The role of paraoxonase 1 activity in cardiovascular disease: potential for therapeutic intervention. Am J Cardiovasc Drugs. 2004; 4(4):211-7; Durrington et al (2001) Paraoxonase and atherosclerosis. Arterioscler Thromb Vasc Biol. 2001 21(4):473-80]. Decreased PON activity has also been found in patients with chronic renal failure, rheumatoid arthritis or Fish-Eye disease (characterized by severe corneal opacities). Hyperthyroidism is also associated with lower serum PON activity, liver diseases, Alzheimer's disease, and vascular dementia. Lower PON activity is also observed in infectious diseases (e.g., during acute phase response). Abnormally low PON levels are also associated with exposure to various exogenous compounds such as environmental chemicals (e.g., metals such as, cobalt, cadmium, nickel, zinc, copper, barium, lanthanum, mercurials; dichloroacetic acid, carbon tetrachloride), drugs (e.g., cholinergic muscarinic antagonist, pravastatin, simvastatin, fluvastatin, alcohol). As mentioned reduced PON levels is also a characteristic of various physiological conditions such as pregnancy, and old age and may be indicative of a subject general health states. For example, smokers exhibit low serum PON1 activity and physical exercise is known to restore PON1 levels in smokers.

Thus, agents (e.g., lactonase substrates such as described hereinabove) of the present invention may be included in a diagnostic kit which may further comprise reaction buffers, storage buffers and sample dilution buffers. Preferably, the kit further comprises a printed matter, such that the printed matter contains instructions of use for the diagnostic kit.

As mentioned hereinabove, the ability to determine the level of biologically active PON may facilitate in determining PON status of an individual.

As used herein the phrase “PON status” refers to PON activity (i.e., lactonase activity) and PON genotype.

Most studies investigating the association of PON1 polymorphism with diseases have examined only nucleotide polymorphism, for which more than 160 polymorphisms have been described including polymorphisms in the coding regions (e.g., Q192R, L55M, C-108T) and in introns and regulatory regions of the gene. However, it has become apparent that even upon genotyping all known PON1 (or others) polymorphisms, this analysis would not provide the level of PON activity nor the phase of polymorphism (i.e., which polymorphisms are on each of an individual's two chromosomes). Thus, functional-genomic analysis will provide a much more informative approach.

Thus, according to another aspect of the present invention there is provided a method of determining PON status of an individual.

The method of this aspect of the present invention is effected by determining lactonase activity level of PON enzymes of the subject, said lactonase activity being indicative of biologically active PON in the subject; and genotyping PON enzymes of the subject, thereby determining PON status of the subject.

Genotyping PON enzymes can be effected at the nucleic acid level or protein level (should the polymorphism affect the translated protein) using molecular biology or biochemical methods which are well known in the art.

Polymorphic forms of PONs may be the result of a single nucleotide polymorphism (SNP), microdeletion and/or microinsertion of at least one nucleotide, short deletions and insertions, multinucleotide changes, short tandem repeats (STR), and variable number of tandem repeats (VNTR).

To obtain polymorphic data, a biological sample comprising the PON enzymes of the subject [e.g., serum sample, urine sample, synnovial fluid sample, biopsy (e.g., hepatic biopsy)] is subjected to allelic determination of DNA polymorphisms, RNA polymorphisms and/or protein polymorphisms.

Following is a non-limiting list of polymorphism (e.g., SNP) detection methods which can be used in accordance with the present invention.

Allele specific oligonucleotide (ASO): In this method an allele-specific oligonucleotides (ASOs) is designed to hybridize in proximity to the polymorphic nucleotide, such that a primer extension or ligation event can be used as the indicator of a match or a mis-match. Hybridization with radioactively labeled allelic specific oligonucleotides (ASO) also has been applied to the detection of specific SNPs (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles.

Pyrosequencing™ analysis (Pyrosequencing, Inc. Westborough, Mass., USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated.

Acycloprime™ analysis (Perkin Elmer, Boston, Mass., USA): This technique is based on fluorescent polarization (FP) detection. Following PCR amplification of the sequence containing the SNP of interest, excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the SNP site. The terminator(s) added are identified by their increased FP and represent the allele(s) present in the original DNA sample. The Acycloprime process uses AcycloPol™, a novel mutant thermostable polymerase from the Archeon family, and a pair of AcycloTerminators™ labeled with R110 and TAMRA, representing the possible alleles for the SNP of interest. AcycloTerminator™ non-nucleotide analogs are biologically active with a variety of DNA polymerases. Similarly to 2′,3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutant have for derivatized 2′,3′-dideoxynucleotide terminators.

It will be appreciated that advances in the field of SNP detection have provided additional accurate, easy, and inexpensive large-scale SNP genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci USA. 88: 7276-80), as well as various DNA “chip” technologies such as the GeneChip microarrays (e.g., Affymetrix SNP chips) which are disclosed in U.S. Pat. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S, and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi MM. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) and MassArray (Leushner J, Chiu N H, 2000. Mol. Diagn. 5: 341-80).

As is mentioned hereinabove, the genetic profile of the cells can also be effected via analysis of cell transcriptomes.

The expression level of the RNA in the cells of the present invention can be determined using methods known in the arts.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

Expression and/or activity level of proteins expressed in the cells of the cultures of the present invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, N.Y.; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., N.Y. (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Synthesis of 5-thioalkyl substituted butyrolactones (TXBL)

The method of synthesis of 4-phenylthio-4-butanolide^{[12]} was used for the synthesis of 5-thioethyl, thiobutyl and thiohexyl butyrolactones (Scheme 2). First, γ-butyrolactone ring was opened with the corresponding thiol^{[13]}. The resulting 4-(alkylthio)-butyric acid was then oxidized with sodium periodate to give 4-(alkylsulfinyl)-butyric acid^{[14]} that was closed to the corresponding lactone by a Pummerer rearrangement^{[12]}. This route was found generic to allow the attachment of side chains of variable length (represented by R in Scheme 3 below) to 5-thio-butyrolactone.

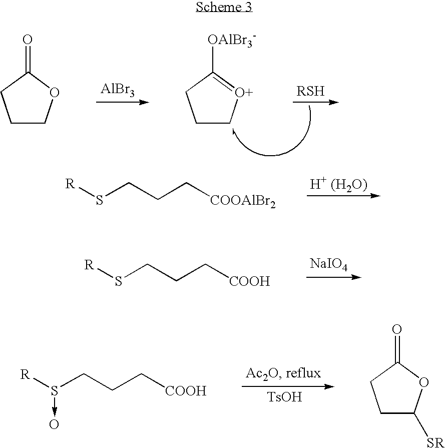

Materials and Experimental Procedures

Materials—Chemicals were purchased from Aldrich Chemicals Co., Fluka and Acros Chemicals.

Typical Synthesis of 5-thioalkyl substituted butyrolactones, Given for 5-thiobutyl butyrolactone (TBBL):

4-(butylthio)-butyric acid. γ-butyrolactone (12.9 mmol, 1.11 gram) was added dropwise to a mixture of AlBr₃ (2.2 eq., 28.38 mmol, 7.56 grams) and butanethiol (about 20 ml). The resulting mixture was stirred 2 hours at room temperature, and then slowly poured on water (about 50 ml). The aqueous mixture was extracted with CH₂Cl₂ (2×50 ml), and the organic phase was washed with NaCl brine, dried over Na₂SO₄. The solvents were evaporated and the product was dried on vacuum. Yield: 1.84 gram, 80.9%.

¹H NMR (250 MHz, CDCl₃): δ (ppm)=0.89-0.94 (t, 3H), 1.36-1.50 (m, 2H), 1.53-1.62 (m, 2H), 1.86-1.97 (m, 2H), 2.46-2.60 (m, 6H).

4-(butylsulfinyl)-butyric acid. To 21 ml (10.5 mmol) of a 0.5 M solution of sodium periodate at 0° C. was added 4-(butylthio)-butyric acid (1.84 gram, 10.4 mmol), and the reaction was stirred overnight at 0° C. The precipitated sodium periodate was removed by filtration, and the filtrate was evaporated. The resulting solid was extracted with CH₂Cl₂ (3×50 ml, 15 minutes extractions), and the solvent was removed by evaporation to yield 4-(butylsulfinyl)-butyric acid (1.88 gram, 94%).

¹H NMR (250 MHz, CDCl₃): δ (ppm)=0.92-0.98 (t, 3H), 1.42-1.53 (m, 2H), 1.68-1.80 (m, 2H), 2.07-2.16 (m, 2H), 2.49-2.64 (t, 2H), 2.69-2.94 (m, 4H).

5-(thiobutyl) butyrolactone. To a solution of 4-(butylsulfinyl)-butyric acid (630 mg, 3.2 mmol) in toluene were added acetic anhydride (3 eq., 10 mmol, 1 gram) and a catalytic amount of p-toluenesulfonic acid. The resulting solution was refluxed for few hours, and the solvents were evaporated to dryness. The residue was dissolved in ethyl acetate:hexane (1:3) and purified by flash chromatography (silica gel, ethyl acetate:hexane (1:3)) to give 5-(thiobutyl) butyrolactone (130 mg, 23.3%).

¹H NMR (400 MHz, CDCl₃): δ (ppm)=0.86-0.92 (t, 3H), 1.40-1.48 (m, 2H), 1.62-1.71 (m, 2H), 2.06-2.18 (m, 2H), 2.49-2.80 (m, 4H), 5.64-5.72 (t, 1H). ¹³C NMR (400 MHz, CDCl₃) δ (ppm): 15.0, 23.3, 29.4, 30.0, 32.8, 33.0, 78.1-79.6. ESI-MS: m/z: 174 [M]⁻.

### Example 2

### Kinetic Analysis of the Enzymatic Hydrolysis of TXBLs

The kinetic parameters of enzymatic hydrolysis of the three TXBLs by PON1 were determined by detecting the released thiol moiety with DTNB.

Materials and Experimental Procedures

Materials—CPM dye (7-diethylamino-3-(4′ maleimidyl-phenyl)-4-methylcoumarin) was purchased from Molecular Probes. Kinetics were performed with recombinant PON1 variant rePON1-G2E6 expressed in fusion with a thioredoxin and 6×His tag, and purified as described^{[19]}.

Kinetic measurements with DTNB—The rates of enzymatic hydrolyses of the thioalkyl-substituted lactones were determined in 50 mM Tris pH 8.0 with 1 mM CaCl₂ and 50 mM NaCl (activity buffer). The enzyme stocks were kept in activity buffer containing 0.1% tergitol, and the enzyme concentration used was 8.375×10⁻⁹ M. Stocks of 100-400 mM of substrates were prepared in acetonitrile and diluted with the reaction buffer immediately before initializing the reaction. 5-(thiohexyl)-butyrolactone (THBL) was dissolved in buffer with Triton X-100 detergent at a final concentration of 0.03-0.24%. The substrate concentrations were varied in the range of 0.3×K_{M} up to (2-3)×K_{M}. The co-solvent percentage was kept at 1% in all reactions. The DTNB dye (Ellman's reagent, 5′,5-dithio bis(2-nitrobenzoic acid) was used from 100 mM stock in DMSO, at a final concentration of 0.5 mM. An ε_{412 nm}=7000 OD/M was used to calculate the activity. Product formation was monitored spectrophotometrically at 412 nm in 200 μl reaction volumes, using 96-well plates, on a microtiter plate reader (PowerWave HT™ Microplate Scanning Spectrophotometer; optical length ˜0.5 cm). Initial velocities (v₀) were determined at eight different concentrations for each substrate. v₀ values were corrected for the background rate of spontaneous hydrolysis in the absence of enzyme. Kinetic parameters (k_{cat}, K_{M}, k_{cat}/K_{M}) were obtained by fitting the data to the Michaelis-Menten equation [v₀=k_{cat}[E]₀[S]₀/([S]₀+K_{M})], using the program Kaleidagraph 5.0.

Kinetic measurements with CPM—The rates of enzymatic hydrolyses of the 4-(thiobutyl) butyrolactone (TBBL) were determined in activity buffer with 8.375×10⁻⁹ M enzyme. The substrate was used from a 400 mM stock in acetonitrile, and it was diluted with the reaction buffer immediately before initializing the reaction and incubated for 3 minutes with the CPM dye (7-diethylamino-3-(4′ maleimidyl-phenyl)-4-methylcoumarin) in order to complete the reaction between CPM and the substrate that was hydrolyzed prior to the measurements. CPM dye was used from 5 mM stock in DMF at final concentration of 50 μM, and the reaction mixtures contained 0.1% triton for CPM solubilization. Product formation was monitored by following the CPM fluorescence in 200 μl reaction volumes, using 96-well plates, on a microtiter plate reader (excitation—400 nm filter, emission—450 and 516 nm filters, Synergy HT™ Multi-Detection Microplate Reader with Time-Resolved Fluorescence; optical length ˜0.5 cm)

Results

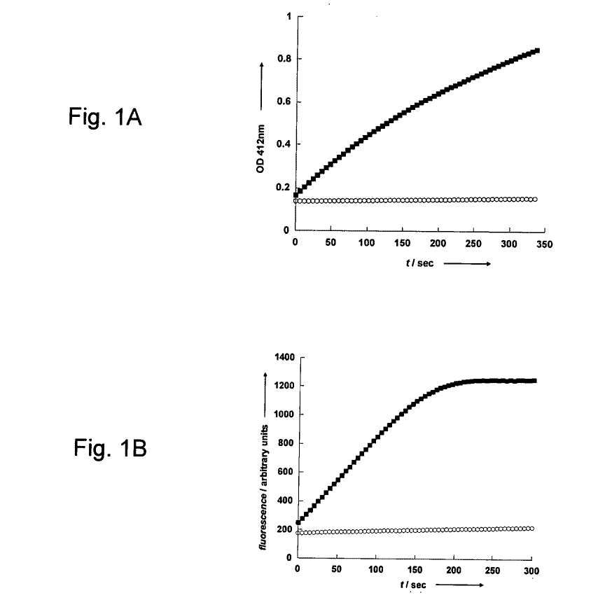

A typical colorimetric assay of 5-(thiobutyl) butyrolactone (TBBL) hydrolysis is shown in FIG. 1a, and the kinetic parameters are listed in Table 1, below. The k_{cat} and K_{M} values for these new substrates are similar to those observed with the homologous 5-alkyl-substituted butyrolactones (Table 2, below).

TABLE 1
Kinetic parameters for rePON1 with S-thioalkyl butyrolactones

| | | k_{cat}, | K_{M}, | k_{cat}/K_{M}, |
|---|---|---|---|---|
| substrate | formula | s⁻¹ | mM | s⁻¹, M⁻¹ |
| TEBL, thioethyl butyrolactone | 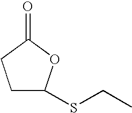 | 161 ± 10 | 0.36 ± 0.05 | 445,000 ± 36,000 |
| TBBL, thiobutyl butyrolactone | 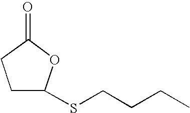 | 116 ± 4 | 0.27 ± 0.04 | 440,000 ± 55,000 |
| THBL, thiohexyl butyrolactone | 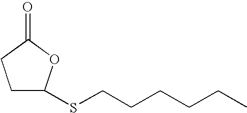 | 52.4 ± 2.6 | 0.35 ± 0.03 | 150,000 ± 9,300 |

TABLE 2
Kinetic parameters for rePON1 with 5-alkyl butyrolactones^{[a]}

| | | k_{cat}, | K_{M,} | k_{cat}/K_{M}, |
|---|---|---|---|---|
| name | structure | s⁻¹ | mM | s⁻¹ M⁻¹ |
| γ-heptanolide | 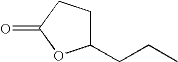 | 34.0 ± 0.8 | 0.58 ± 0.03 | 58,000 ± 3,000 |
| γ-nonanoic lactone | 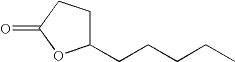 | 31 ± 2 | 0.39 ± 0.03 | 78,000 ± 1,600 |
| γ-undecanoic lactone | 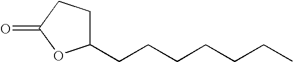 | 62 ± 2 | 0.60 ± 0.07 | 103,000 ± 8,600 |

^{[a]}-The kinetic parameters for 5-alkyl butyrolactones are taken from Ref.^{[4]}

The rates of enzymatic hydrolyses of the 5-thioalkyl lactones were also followed with the fluorogenic thiol detecting probe CPM^{[11]} as shown in FIG. 1b.

### Example 3

### Measurement of PON1 Activity in Human Sera and Living Cells

The above described chromogenic and fluorogenic assays were used for determining lactonase activity of PONs in human serum samples.

Materials and Experimental Procedures

Serum activity with TBBL and phenyl acetate—Reactions were performed in activity buffer, and the serum was used at final dilution of 1 to 400. The reaction mixtures of TBBL contained 0.5 mM TBBL from 400 mM stock in acetonitrile and 0.5 mM DTNB from 100 mM stock in DMSO. The reaction mixtures of phenyl acetate contained 1 mM phenyl acetate from 500 mM stock in methanol. All the reaction mixtures contained final 1% DMSO. 2-hydroxyquinoline was used from 500 mM stock in DMSO, and EDTA was used from 0.5 M stock in water. The serum was incubated with the inhibitors for 5-10 minutes before the initiation of the reaction.

Detection of PON1 activity with TBBL by FACS—The emulsification of the E. Coli cells and FACS analysis were performed as previously described.^{[16]}

Results

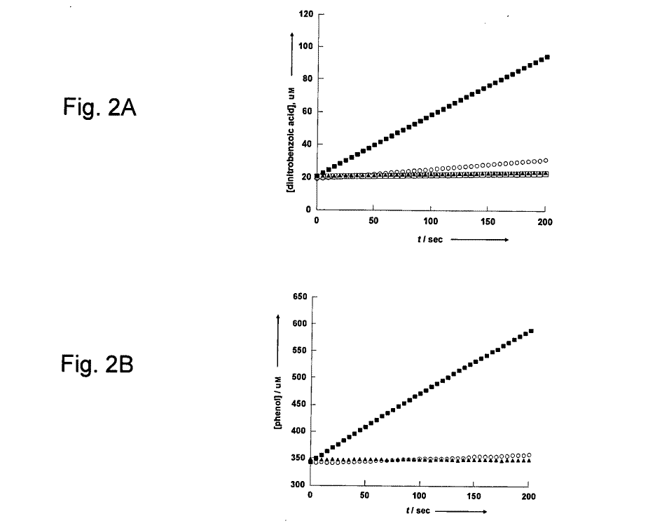

PON1 levels in human sera were detected using the newly synthesized substrates (see Examples 1-2), as demonstrated in FIGS. 2a-b. To verify that the measured lactonase activity is mediated by PON1 as opposed to other hydrolases presence in the serum, the serum was also pre-incubated with 2-hydroxyquinoline (a selective competitive inhibitor of PON1's activity^{[4]}), and EDTA (chelating the calcium which is crucial for PON 1's activity). In parallel, we the PON1 activity was determined with phenyl acetate, which is routinely used as a probe for PON1 levels in the serum. The activity with TBBL was comparable to that with phenyl acetate, and was similarly inhibited (see Table 3 below). This clearly demonstrates that the novel lactone substrates can be used for assessing PON1 levels in human sera, and that >90% of the lactonase and aryl esterase activities stem from PON1. The higher inhibition rates by EDTA (>99%) might be due to serum enzymes other than PON1 that are sensitive to metal chelators.

TABLE 3
Serum activity with phenyl acetate and TBBL

| | Serum activity with 0.5 mM TBBL, | | | Serum activity with 1 mM phenyl | | |
|---|---|---|---|---|---|---|
| | μM product/min | | | acetate, μM product/min | | |
| | (% of uninhibited activity) | | | (% of uninhibited activity) | | |
| | | | 5 mM | | 100 μM | 5 mM |
| Sample # | uninhibited | 100 μM HQ | EDTA | uninhibited | HQ | EDTA |
| 1 | 21.0 ± 0.4 | 1.80 ± 0.01 | 0.06 ± 0.01 | 79 ± 6 | 3.9 ± 0.3 | ~0 |
| | | (8.6%) | (0.3%) | | (4.9%) | (0%) |
| 2 | 21.3 ± 0.1 | 2.09 ± 0.04 | 0.04 ± 0.01 | 80 ± 3 | 5.9 ± 0.4 | ~0 |
| | | (9.8%) | (0.2%) | | (7.4%) | (0%) |

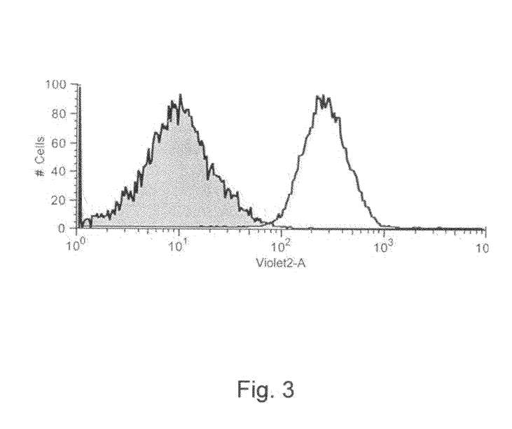

PON1 activity was also detected in living cells, using FACS (fluorescence-activated cell sorter) and emulsion droplets that compartmentalize the cells together with the products of the enzymatic activity^{[15, 16]}. First, E. coli cells expressing recombinant PON1 (rePON1) in cytoplasm, as well as GFP (green fluorescent protein) were compartmentalized in the aqueous droplets of a water-in-oil (w/o) emulsion, together with the lactone substrate (TBBL) and the fluorogenic thiol-detecting dye CPM. The w/o emulsion was then re-emulsified, to generate the w/o/w double emulsion with a continuous water phase that is amenable to FACS^{[15]}. The FACS triggering threshold was set for the emission of GFP, and an appropriate gate was chosen corresponding to the level of emission of single E. coli cells^{[16]}. As shown in FIG. 3, the detection of PON1 lactonase activity in the compartmentalized cells was via the fluorescent signal of the thiol-detecting dye at 530 nm. A clear difference (>20-fold in mean fluorescence) was observed relative to cells bearing no rePON1

In conclusion, the above-results demonstrate that 5-thioalkyl lactones are highly useful and sensitive probes for assaying the lactonase activity of PON1. The rates of PON1 with these substrates are similar to aliphatic 5-alkyl substituted lactones that are favorable substrates of PON1 and may well resemble its native substrates^{[2]}. The 5-thioalkyl lactones can be used with complex biological samples such as intact cells and sera, and thus provide a novel, physiologically relevant mean of testing the levels of PON1 in human serum in a high-throughput manner. These substrates also provide a powerful mean of screening for lactonase activity using FACS and double emulsions, that enable the screen of libraries of >10⁷ enzyme variants in few hours, for directed evolution and functional genomics^{[16, 17]}. Finally, the novel 5-thioalkyl lactones can be used with enzymes other than PON1, in particular with other PON family members for which no chromogenic/fluorogenic substrates exist. For example, the lactonase activity of PON3 could be assayed with TEBL and TBBL, both in purified enzyme samples and crude cell lysates (data not shown). The lactonase activity of other enzymes (e.g., Pseudomonas diminuta phosphotriesterase) could also be detected^{[18]}.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

### REFERENCES CITED BY NUMERALS

### Other References are Cited in the Document

[1] S. Billecke, D. Draganov, R. Counsell, P. Stetson, C. Watson, C. Hsu, B. N. La Du, Drug Metab. Dispos. 2000, 28, 1335.
[2] D. I. Draganov, J. F. Teiber, A. Speelman, Y. Osawa, R. Sunahara, B. N. La Du, J Lipid Res. 2005, 46, 1239.
[3] J. F. Teiber, D. I. Draganov, B. N. La Du, Biochem. Pharmacol. 2003, 66, 887.
[4] 0. Khersonsky, D. S. Tawfik, Biochemistry 2005, 44, 6371.
[5] A. Aharoni, L. Gaidukov, O. Khersonsky, S. McQ. Gould, C. Roodveldt, D. S. Tawfik, Nat. Genet. 2005, 37, 73.
[6] L. Gaidukov, D. S. Tawfik, Biochemistry 2005, in press.
[7] L. G. Costa, A. Vitalone, T. B. Cole, C. E. Furlong, Biochem. Pharmacol. 2005, 69, 541.
[8] J. P. Goddard, J. L. Reymond, Trends Biotechnol. 2004, 22, 363.
[9] R. Sicard, L. S. Chen, A. J. Marsaioli, J. L. Reymond, Adv. Synth. Catal. 2005, 347, 1041.
[10] G. L. Ellman, Arch. Biochem. Biophys. 1958, 74, 443.
[11] R. P. Haugland, in Handbook of Fluorescent Probes and Research Products 9th ed., Molecular Probes, Eugene, 2002, p. 79.
[12] M. Watanabe, S, Nakamori, H. Hasegawa, K. Shirai, T. Kumamoto, Bull. Chem. Soc. Japan 1981, 54, 817.
[13] M. Node, K. Nishide, M. Sai, E. Fujita, Tetrahedron Lett. 1978, 52, 5211.
[14] N. J. Leonard, C. R. Johnson, J. Org. Chem. 1961, 27, 282.
[15] K. Bernath, M. T. Hai, E. Mastrobattista, A. D. Griffiths, S. Magdassi, D. S. Tawfik, Analytical Biochemistry 2004, 325, 151.
[16] A. Aharoni, G. Amitai, B. Bernath, S. Magdassi, D. S. Tawfik, Submitted for publication 2005.
[17] A. Aharoni, A. D. Griffiths, D. S. Tawfik, Curr. Opin. Chem. Biol. 2005, 9, 210.
[18] C. Roodveldt, D. S. Tawfik, Biochemistry 2005, in press.
[19] A. Aharoni, L. Gaidukov, S. Yagur, L. Toker, I. Silman, D. S. Tawfik, Proc. Natl. Acad. Sci. USA 2004, 101, 482.

1. A method of determining a level of biologically active PON enzyme, the method comprising determining lactonase activity of the PON enzyme, said lactonase activity being indicative of the level of biologically active PON enzyme.

2. A method of determining PON status in a subject, the method comprising:
(a) determining lactonase activity level of a PON enzyme of the subject, said lactonase activity being indicative of the level of biologically active PON in the subject; and
(b) genotyping said PON enzymes of the subject, thereby determining PON status of the subject.

3. The method of claim 1, wherein the PON enzyme is selected from the group consisting of PON1, PON2 and PON3.

4. The method of claim 1, wherein said biologically active PON enzyme comprises apolipoprotein complexed PON enzyme.

5. The method of claim 1, wherein determining lactonase activity of the PON enzyme is effected by:
(i) a chromatographic analysis;
(ii) a pH indicator assay;
(iii) a spectrophotometric assay;
(iv) a coupled assay;
(v) an electrochemical assay; and/or
(vi) a therm-ocalometric assay.

6. The method of claim 5, wherein said spectrophotometric assay is effected in the presence of a substrate comprising at least one lactone and being capable of forming at least one spectrophotometrically detectable moiety upon hydrolysis of said lactone.

7. The method of claim 5, wherein said spectrophotometric assay is selected from the group consisting of a phosphorescence assay, a fluorescence assay, a chromogenic assay, a luminescence assay and an illuminiscence assay.

8. The method of claim 6, wherein said detectable moiety is attached to said lactone.

9. The method of claim 6, wherein said detectable moiety forms a part of said lactone.

10. The method of claim 6, wherein said detectable moiety comprises at least one thiol.

11. The method of claim 10, wherein said substrate comprises a thioalkoxy group being attached to said lactone.

12. The method of claim 11, wherein said thioalkoxy group comprises from 2 to 12 carbon atoms.

13. The method of claim 10, wherein said detecting is effected by a chromogenic assay or a fluorogenic assay.

14. The method of claim 6, wherein said substrate comprises a 5-, 6- or 7-membered lactone having a thioalkoxy group attached to the carbon adjacent to the heteroatom of said lactone.

15. A method of determining activity of a lactonase in a sample comprising:
(a) contacting the sample with a compound containing at least one lactone and being capable of forming at least one spectrophotometrically detectable moiety upon hydrolysis of said lactone, wherein said detectable moiety is selected such that said compound has substantially the same structure as a substrate of said lactonase; and
(b) spectrophotometrically measuring a level of said moiety, thereby determining an activity of the lactonase in the sample.

16. The method of claim 15, wherein measuring said level of said moiety is effected by a phosphorescence assay, a fluorescence assay, a chromogenic assay, a luminescence assay and an illuminiscence assay.

17. The method of claim 15, wherein said detectable moiety is attached to said lactone.

18. The method of claim 15, wherein said detectable moiety forms a part of said lactone.

19. The method of claim 15, wherein said detectable moiety comprises at least one thiol.

20. The method of claim 19, wherein said substrate comprises a thioalkoxy group being attached to said lactone.

21. The method of claim 20, wherein said thioalkoxy group comprises from 2 to 12 carbon atoms.

22. The method of claim 19, wherein said detecting is effected by a chromogenic assay.

23. A kit for determining predisposition or diagnosing a disorder associated with abnormal levels or activity of a PON enzyme in a subject, the kit comprising at least one agent capable of determining lactonase activity of the PON enzyme.

24. The kit of claim 23, wherein said at least one agent is a compound comprising at least one lactone and being capable of forming at least one spectrophotometrically detectable moiety upon hydrolysis of said lactone.

25. A compound comprising at least one lactone and being capable of forming at least one spectrophotometrically detectable thiol-containing moiety upon decomposition of said lactone.

26. The compound of claim 25, wherein said thiol-containing moiety is detectable by a spectrophotometric assay selected from the group consisting of a phosphorescence assay, a fluorescence assay, a chromogenic assay, a luminescence assay and an illuminiscence assay.

27. The compound of claim 25, wherein said detectable moiety is attached to said lactone.

28. The compound of claim 25, wherein said detectable moiety forms a part of said lactone.

29. The compound of claim 26, wherein said detectable moiety comprises a thioalkoxy group.

30. The compound of claim 29, wherein said thioalkoxy group comprises from 2 to 12 carbon atoms.

31. The compound of claim 27, wherein said lactone is a 5-, 6- or 7-membered lactone.

32. The compound of claim 27, wherein said lactone is a five-membered lactone.

33. The method of claim 2, wherein the PON enzyme is selected from the group consisting of PON1, PON2 and PON3.

34. The method of claim 2, wherein said biologically active PON enzyme comprises apolipoprotein complexed PON enzyme.

35. The method of claim 2, wherein determining lactonase activity of the PON enzyme is effected by:
(i) a chromatographic analysis;
(ii) a pH indicator assay;
(iii) a spectrophotometric assay;
(iv) a coupled assay;
(v) an electrochemical assay; and/or
(vi) a therm-ocalometric assay.
